(19) United States
(12) Patent Application Publication
(10) Pub. No.: US 2015/0277015 A1
(43) Pub. Date: Oct. 1, 2015

(54) LIGHT GUIDE TUBE

(71) Applicants: Luke Lu, South Pasadena, CA (US); Toshihiro Tokizawa, Tokyo (JP); Kuan-Yu Lu, Taipei City (TW); I-Ching Lu, Taipei (TW)

(72) Inventors: Luke Lu, South Pasadena, CA (US); Toshihiro Tokizawa, Tokyo (JP); Kuan-Yu Lu, Taipei City (TW); I-Ching Lu, Taipei (TW)

(21) Appl. No.: 14/228,272

(22) Filed: Mar. 28, 2014

(51) Int. Cl. F21V 8/00; A61B 1/00

(52) U.S. Cl. CPC G02B 6/0005; A61B 1/00165

(57) ABSTRACT

The present invention relates to a light guide tube, which is formed by weaving a plurality of filaments to provide a single-hole or multi-hole tube. Further, the filaments include filaments that are made of at least one light-transmittable material. With the single-hole or multi-hole tube formed by weaving filaments, besides strengthening the capability of enduring sideways stress, a light-emitting unit can be additionally included at a front end of the tube for illumination purposes and formation can be achieved by using various types of filaments to suit practical needs, such as combination with metal wires or electrical signal carrier material for purposes of transmission of electrical power or electronic information, thereby achieving a wider range of applications that the conventional tubes.

### (a) TECHNICAL FIELD OF THE INVENTION

The present invention generally relates to a tubular article, and more particularly to a light guide tube that is formed by weaving filaments.

### (b) DESCRIPTION OF THE PRIOR ART

The progress of science and technology provides conveniences of people's living and also improve medical techniques, particularly the invasive surgical operations that require a surgeon or a professional person to operate dedicated equipment to intrude human body or other biological tissues to remove lesion, change structure, or implant a foreign object through the application of an external measure.

The size of an incision created in a surgical operation is of definite influence on the after-operation recovery. In view of this, the minimally invasive surgery is one of the currently developed items. There are more and more surgical operations that can invade human body through mouth, nose, anus, or incision of skin to enable equipment to insert into the human body for operation under the guide of a surgeon to carry out cutting or sewing operations. However, the equipment used to carry out the operations is often carried by a tubular article to reach into the human body. Further, tubular articles are also used for pumping body fluid, illumination, or photographing.

On the other hands, there are a number of electrical or mechanical piping systems that are very complicated. To identify a malfunctioning site, a tubular article, which is similar to an endoscope, is used to carry various equipment to reach into the interior of machines for observation and identification.

The above discussion indicates that the tubular articles have a variety of applications. Particularly, tubes can be used with various types of equipment to carry out liquid pumping or to carry equipment to get into human body or the interior of a machine for inspection. However, all sorts of equipment are being improved from time to time and the tube, although being a single piece of accessory, has been under development in respect of the material thereof, but none is focused on modification and improvement of the structure thereof. Thus, for the technical field of tube, further improvement is necessary.

### SUMMARY OF THE INVENTION

In light of the above discussed, the present invention aims to provide a light guide tube, which is formed by weaving a plurality of filaments to provide a single-hole or multi-hole light guide tube and comprises filaments made of at least one light-transmittable material. With the light guide tube formed by weaving filaments, besides strengthening the capability of enduring sideways stress, a use for transmitting light can be realized with light emitting from an outlet or other sites of the light guide tube for illumination purposes so that the range of application is made wider.

Preferably, the filaments are coupled to resin to form a unitary structure.

Preferably, the filaments further comprise a glass fiber material, a carbon fiber material, a ceramic fiber material, a glass-ceramic material, a metal material, or a plastic fiber material.

Further, the filaments of light-transmittable material can be single core optic filaments or compound optic filaments, manufacturing and combination being made to suit different needs.

Further, the compound optic filaments comprise resin surrounding a light-transmitting material and the resin has a refractive index lower than a refractive index of the light-transmitting material.

Preferably, the resin can be thermoplastic resin or thermosetting resin.

Preferably, the resin has a refractive index that is lower than a refractive index of one of the filaments.

The foregoing objectives and summary provide only a brief introduction to the present invention. To fully appreciate these and other objects of the present invention as well as the invention itself, all of which will become apparent to those skilled in the art, the following detailed description of the invention and the claims should be read in conjunction with the accompanying drawings. Throughout the specification and drawings identical reference numerals refer to identical or similar parts.

Many other advantages and features of the present invention will become manifest to those versed in the art upon making reference to the detailed description and the accompanying sheets of drawings in which a preferred structural embodiment incorporating the principles of the present invention is shown by way of illustrative example.

### BRIEF DESCRIPTION OF THE DRAWINGS

FIG. 1 is a perspective view showing an embodiment of the present invention.

FIG. 2 is a cross-sectional view of the embodiment of the present invention.

FIG. 3 is a cross-sectional view showing another embodiment of the present invention.

FIG. 4 is a cross-sectional view showing a multi-hole tube according to an embodiment of the present invention.

FIG. 5 is a cross-sectional view showing a multi-hole tube according to another embodiment of the present invention.

FIG. 6 is an enlarged view showing the portion A of FIG. 1 according to an embodiment of the present invention.

FIG. 7 is an enlarged view showing the portion A of FIG. 1 according to another embodiment of the present invention.

FIG. 8 is an enlarged view showing the portion A of FIG. 1 according to a further embodiment of the present invention.

FIG. 9 is a perspective view showing another embodiment of the present invention.

FIG. 10 is a perspective view showing a further embodiment of the present invention.

FIG. 11 is a perspective view showing a light-transmittable filament used in an embodiment of the present invention.

FIG. 12 is a perspective view showing a light-transmittable filament used in an embodiment of the present invention.

FIG. 13 is a cross-sectional view of a filament shown in FIG. 10.

FIG. 14 is a perspective view showing a light-transmittable filament used in another embodiment of the present invention.

### DETAILED DESCRIPTION OF THE PREFERRED EMBODIMENTS

The following descriptions are exemplary embodiments only, and are not intended to limit the scope, applicability or configuration of the invention in any way. Rather, the following description provides a convenient illustration for implementing exemplary embodiments of the invention. Various changes to the described embodiments may be made in the function and arrangement of the elements described without departing from the scope of the invention as set forth in the appended claims.

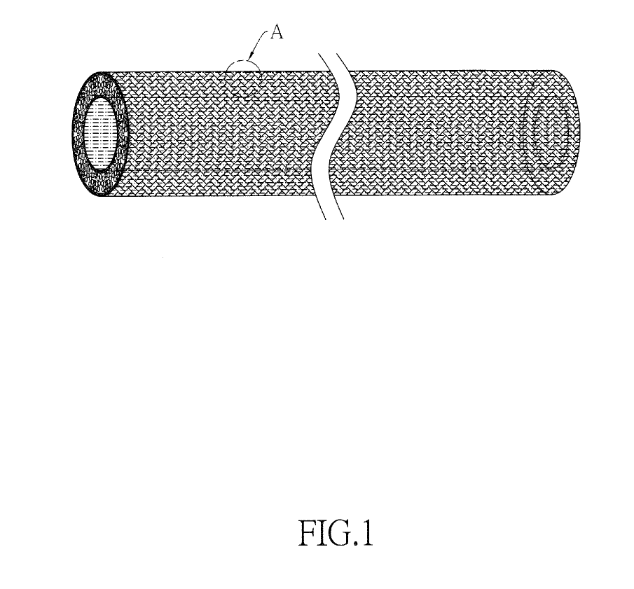

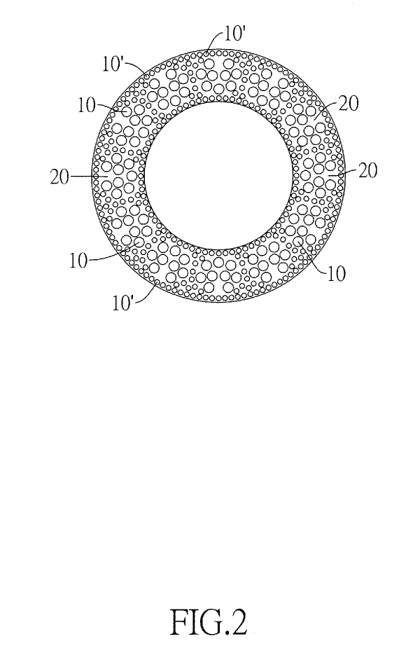

Referring to FIGS. 1 and 2, the instant embodiment is a light guide tube, which is formed by weaving a plurality of filaments 10. The filaments 10 include filaments made of at least one light-transmittable material. For the light guide tube formed by weaving the filaments 10, the light guide tube can bear a relatively large shear stress and allows light to be transmitted through the filaments of the light-transmittable material, whereby improved flexibility is provided and an output terminal of the filaments of the light-transmittable material can be set at an end of the tube or in a body of the tube for illumination purposes. Further, the light guide tube of the instant embodiment can be directly coupled to an optic lens for image displaying or photographing purposes so as to show wider range of applications than those of the conventional tubes.

Further, after completion of the weaving operation, resin 20 is coupled to the outside of all the filaments 10 for easing subsequent operations, such as cleansing. Further, the resin 20 can be thermoplastic resin or thermosetting resin or a resin mixture that is resistant to high temperature and high pressure.

Further, the filaments 10 can be a light-transmittable material, a glass fiber material, a carbon fiber material, a ceramic fiber material, a glass-ceramic fiber material, a metal material, an electrical signal carrier material, or a plastic fiber material. Through the use of various materials, combinations thereof can be made to provide tubes of any desired property, such as raising hardness, increasing elasticity, providing capability of signal transmission, or providing capability of transmission of electrical power, so as to improve the convenience of use.

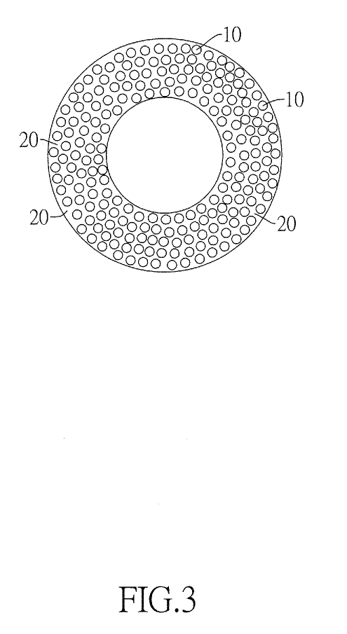

Further, referring to both FIGS. 2 and 3, FIG. 2 shows that the filaments 10, 10′ have more than two cross-section diameters, but in FIG. 3, the filaments 10 have only one size. The difference of the two is determined according to various materials or according to the needs to provide filaments of corresponding sizes. Thus, it can be appreciated from the two drawings that there is generally of no constraint on the diameter and arrangement of the filaments used in the present invention. For example, the filaments 10′ in a circumferential portion can be made of a metal material to increase the strength of the circumferential portion of the tube or can be made of a light-transmittable material for the purposes of transmitting light.

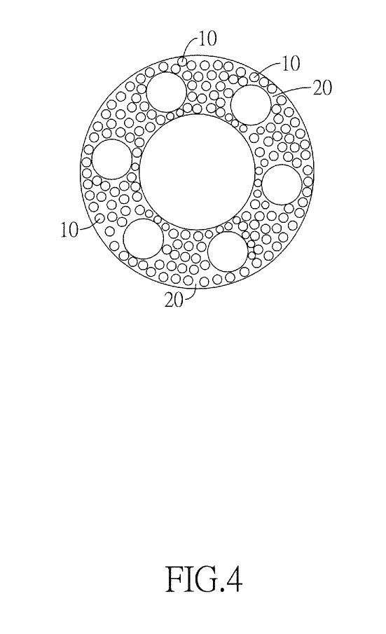

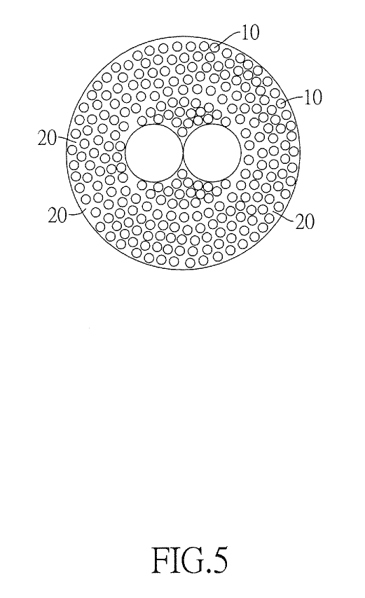

Further, referring to various cross-sectional shapes illustrated in FIGS. 3-5, FIG. 3 shows a cross-section of a single-hole configuration. FIG. 4 shows a tube having a multi-hole configuration for disposition therein a number of devices or receiving therein piano wires (or steel wires) that are fixed to one end of the holes, so that pulling the piano wires can control the direction of flexing of the tube. Further, in FIG. 5, a tube of a two-hole configuration is illustrated. It can be appreciated from these three drawings that the instant embodiment provide a tube of any shape through weaving and can be arranged for combining, connecting, splitting channels in the tube so as to meet various needs.

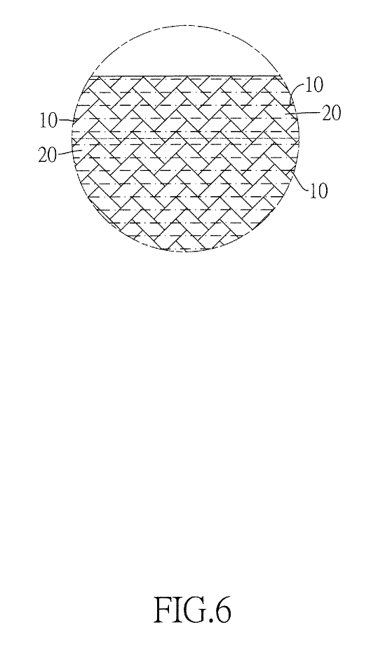

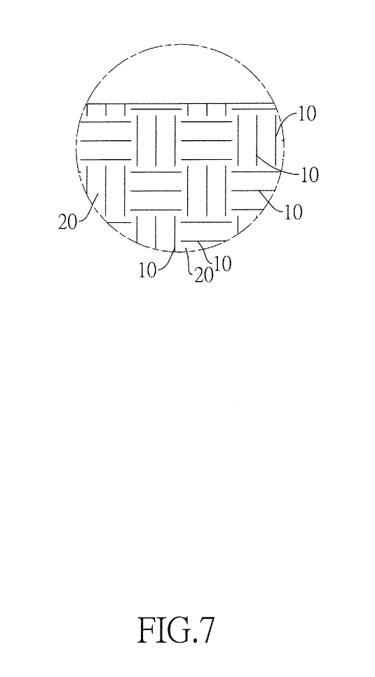

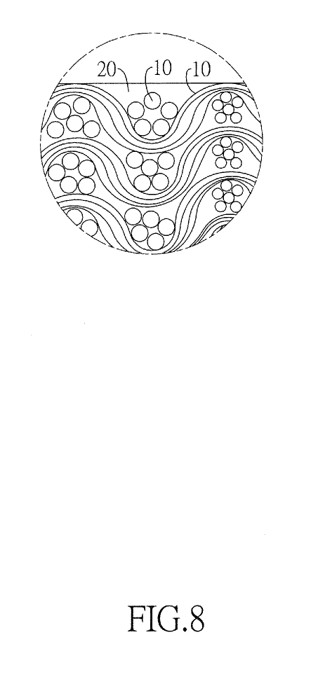

Further, as shown in FIG. 6, which is an enlarged view of portion A of FIG. 1, a form of inter-weaving of the filaments 10 is illustrated. Alternatively, as shown in FIG. 7, several of the filaments 10 can be collected as a group and are woven to show a perpendicularly intersected arrangement. Or alternatively, a further form of weaving and intersecting arrangement is shown in FIG. 8. It can be appreciated from these drawings that numerous ways of weaving can be used and different ways of weaving are selected to meet different needs or the properties of different materials in order to provide a desired effectiveness and are not limited to what shown in the drawings. Further, some of the filaments are arranged to have two ends thereof located at opposite ends of the light guide tube (a sort of extending through) to combine with filaments of identical or different lengths that are arranged circumferentially for weaving and forming the light guide tube.

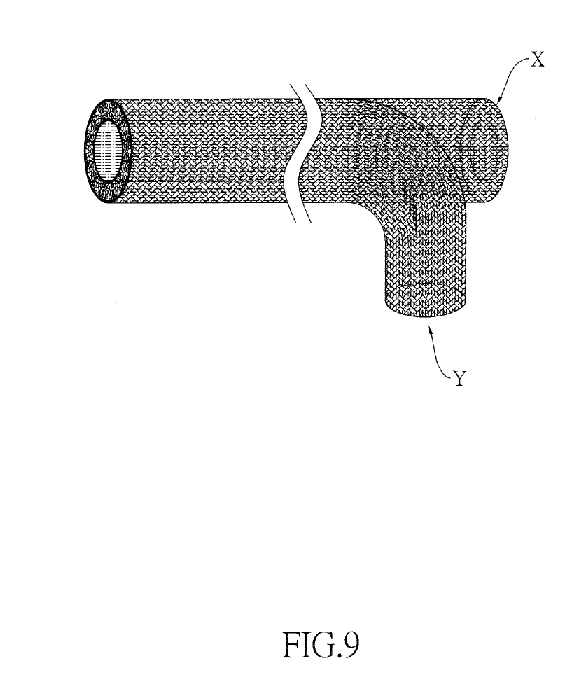

As shown in FIG. 9, filaments of the light-transmittable material are woven at an end X of the light guide tube, while the hollow holes of the light guide tube are formed at another end Y. The light guide tube so illustrated enables connection of a light-emitting element to the end X to allow light to transmit through the filaments of the light-transmittable material to a tube opening or to a specific site for projection of light. Another end Y of the light guide tube can be connected to a pumping device to enable the use of the light guide tube for pumping liquid. As such, a single light guide tube can be used for both illumination and pumping so that the convenience of use can be greatly enhanced.

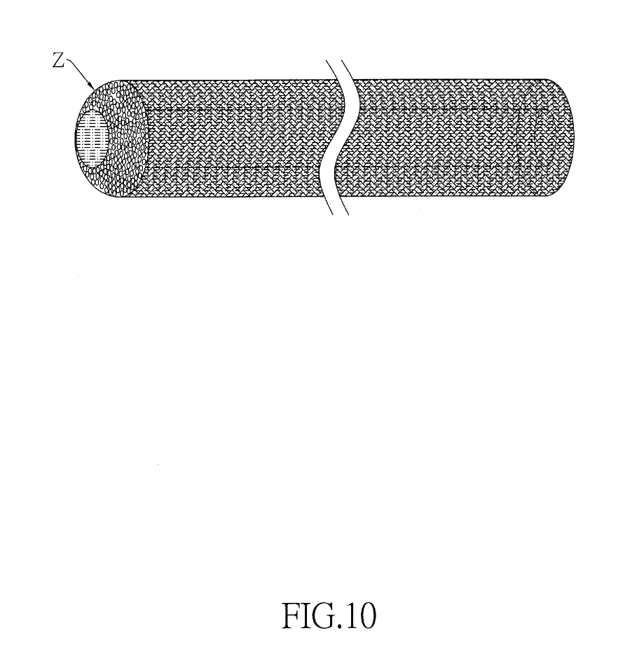

FIG. 10 shows that an end of the light guide tube can be made as a convex curved surface Z to meet the needs and is thus not limited to a flat end face.

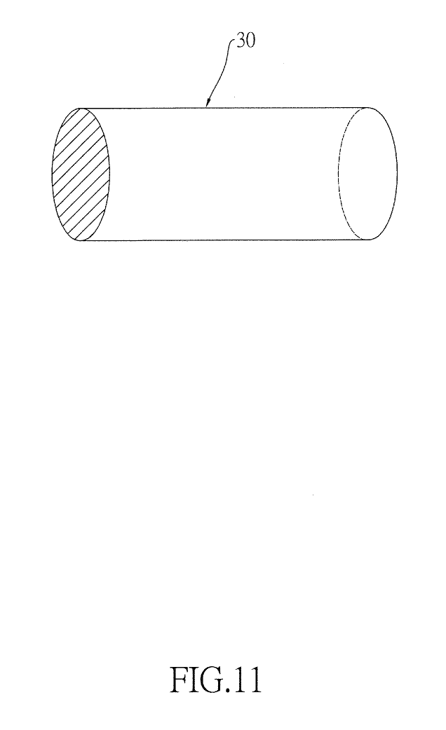

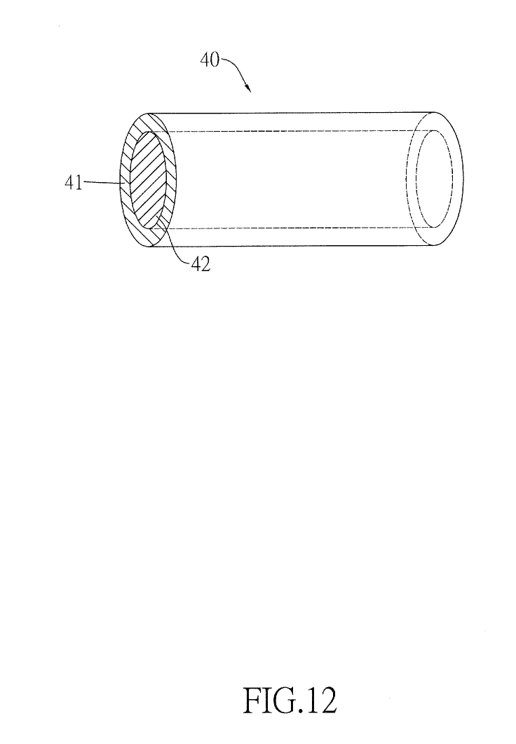

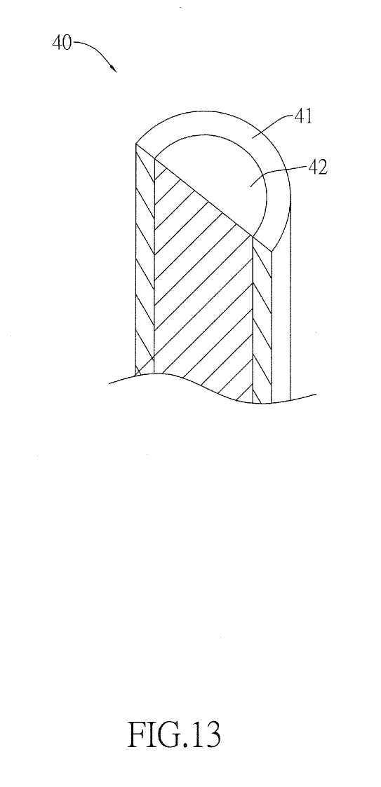

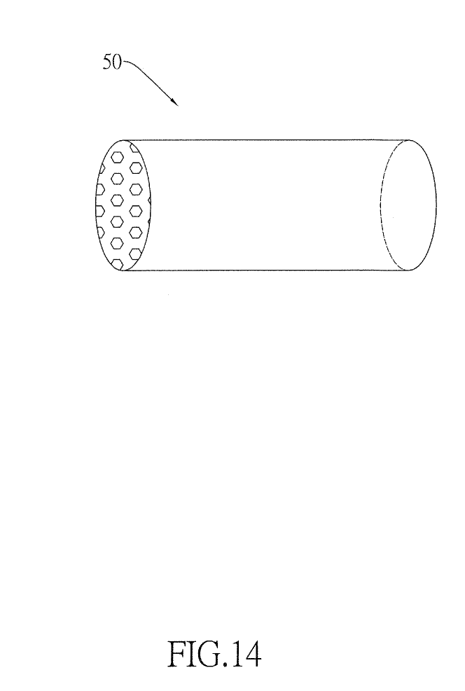

Further, the filaments 10 of light-transmittable material can be a single core optic filament 30 (as shown in FIG. 11) or a compound optic filament 40 (as shown in FIGS. 12 and 13) or a multi-core optic filament 50 shown in FIG. 14. There can be numerous types of filaments 10 of light-transmittable material or materials of different refractive indexes can be combined to form the light-transmittable material. Further, the compound optic filament 40 can be composed of resin 41 coupled to a light transmitting layer 42, wherein the resin 41 has a refractive index lower than the refractive index of the light-transmitting layer 42. (The multi-core optic filament 50 can be manufactured in the same way with the only difference being the number of the light-transmitting layers.) Referring to FIG. 2, the filaments 10′ of the circumferential portion can be made according to what described in this paragraph, where the light-transmitting layer is coupled to resin 20 having lower refractive index to achieve the purposes of transmitting light.

There are a number of materials for transmitting light and also a number of light-permeable materials. They are not constrained by the embodiments described herein and all the materials that can transmit light are considered equivalent materials of the present invention.

It will be understood that each of the elements described above, or two or more together may also find a useful application in other types of methods differing from the type described above.

While certain novel features of this invention have been shown and described and are pointed out in the annexed claim, it is not intended to be limited to the details above, since it will be understood that various omissions, modifications, substitutions and changes in the forms and details of the device illustrated and in its operation can be made by those skilled in the art without departing in any way from the spirit of the present invention.

I claim:

1. A light guide tube, which is formed by weaving a plurality of filaments, wherein the filaments include filaments that are made of at least one light-transmittable material.

2. The light guide tube according to claim 1, wherein an outside of the filaments is coupled to resin.

3. The light guide tube according to claim 2, wherein the filaments further comprise a glass fiber material, a carbon fiber material, a ceramic fiber material, a glass-ceramic material, a metal material, an electrical signal carrier material, or a plastic fiber material.

4. The light guide tube according to claim 1, wherein the filaments of light-transmittable material comprise single core optic filaments or compound optic filaments.

5. The light guide tube according to claim 4, wherein the compound optic filaments comprise resin coupled to a light-transmitting material, the resin having a refractive index lower than a refractive index of the light-transmitting material.

6. The light guide tube according to claim 2, wherein the resin comprises a thermoplastic resin.

7. The light guide tube according to claim 2, wherein the resin has a refractive index lower than a refractive index of one of the filaments.

8. The light guide tube according to claim 1, wherein the light guide tube comprises a plurality of through holes.
